(19) United States
(12) Patent Application Publication
(10) Pub. No.: US 2023/0393040 A1
(43) Pub. Date: Dec. 7, 2023

(54) Method and System of Sample Pooling and Processing

(71) Applicant: Kui Gao, Poway, CA (US)

(72) Inventor: Kui Gao, Poway, CA (US)

(21) Appl. No.: 18/033,254

(22) PCT Filed: Oct. 21, 2021
(86) PCT No.: PCT/US2021/056082

§ 371 Date: Apr. 21, 2023

Related U.S. Application Data
(60) Provisional application No. 63/197,707, filed on Jun. 7, 2021.
(60) Provisional application No. 63/104,207, filed on Oct. 22, 2020.

(51) Int. Cl. G01N 1/38

(52) U.S. Cl. CPC G01N 1/38

(57) ABSTRACT

The present disclosure includes, but is not limited to, methods and systems for pooling or combining multiple samples together. Particularly, this disclosure relates to pooling and combining samples in a process and system that combines samples in a way that allows targets in the pooled sample to be analyzed/detected with a similar sensitivity and specificity as for analysis/detection of targets from the individual samples of the pool. Specifically, in some embodiments, methods herein avoid a dilution effect found in commonly used sample pooling methods that can reduce their sensitivity and limit the number of samples that can be pooled. In some embodiments, methods herein include processes to de-convolute the pool down to individual samples. Methods herein may be applicable to creating and testing pooled/combined samples for target molecules and de-convoluting pooled samples to identify an individual sample in a pool containing the target. Systems for performing the methods, including in some embodiments partially or fully automated methods, are also described.

### CROSS-REFERENCE TO RELATED APPLICATIONS

This application claims priority to U.S. Provisional Application No. 63/104,207, filed on Oct. 22, 2020, and to U.S. Provisional Application No. 63/197,707, filed on Jun. 7, 2021, the disclosures of which are hereby incorporated by reference in their entireties.

### FIELD OF THE DISCLOSURE

This disclosure provides methods for pooling individual samples to maintain the performance of downstream analysis and detection without loss of sensitivity due to dilution effect by pooling. The methods comprise pooling specimens from multiple individuals; different specimen types from same individual or any combination of specimens from subjects. The disclosure also provides systems for performing the methods.

### BACKGROUND

A conventional pooling method comprises, for example, combining small aliquots of samples from multiple individual samples into a pooled sample. An analytical or diagnostic test is conducted on the pooled samples rather than on the individual samples in order, for example, to save time, testing supplies, and labor. (See, e.g., FIGS. 1 and 2.) Sample pooling, however, creates a dilution effect that can reduce the sensitivity of a target detection assay. For example, if only one sample out of five contains the target molecule, as shown in FIG. 2, pooling all five samples will effectively dilute the target 5-fold, making it potentially harder to detect. Similarly, testing of pooled samples from 8 individual samples can reduce the sensitivity of target detection by a factor of 8 compared to testing the individual samples one by one. In spite of this concern, this conventional sample pooling and pool testing approach has been suggested and applied to pathogen screening of blood samples for transfusions and to COVID-19 population screening, for example. (See, e.g., V. Shyamala, Vox Sang. 9(2): 315-324 (2014); N. Agarwal et al., Asian J. Transfusion Science 8(1): 26 (2014); A. Fogarty et al., Lancet Resp. Med. 8(11): P1078-80 (Sep. 22, 2020); J. Garg et al., J. Med. Virology 93(3): 1526-31 (2020).)

Millions of blood donations may need to be tested annually for pathogen contamination, prior to being cleared for use in transfusion, for example. For detection of pathogenic nucleic acid sequences, either individual samples or pooled samples can be tested. Individual donor testing is ideal methodology since dilution due to pooling reduces the sensitivity of the detection and can lead to improperly clearing samples that have low concentration of pathogens. Nevertheless, pool testing has been applied to increase the test efficiency and to reduce the cost. However, the reduced sensitivity, due to pooling, potentially increases the risk of transfusion transmissions and can cause a blood safety issue. It has been considered impossible for testing pooled samples to achieve the same sensitivity as testing individual samples. (See V. Shyamala, above.)

On Mar. 11, 2020, the World Health Organization (WHO) declared the pandemic of COVID-19. As of Oct. 15th 2020, there have been 38,394,169 confirmed cases of COVID-19 and 1,089,047 confirmed deaths, globally reported to WHO. Many countries, including the U.S., during the COVID-19 pandemic have experienced shortages in testing kits, and testing supplies, as well as licensed laboratory personnel to perform testing. Pool testing was proposed and encouraged by the US FDA to increases the number of individuals that can be tested using the same amount of resources. (See, e.g., C. R. Weinberg, Am. J. Epidemiology, 189(5): 363-364 (2020); N. Lagonati et al., J. Vir. Meth. 289, p. 114044 (2021); B. Abdalhamid et al., Am. J. Clin. Pathol. 153: 715-718 (2020).) For example, as of October 2020, five (such as Aptima SARS-CoV-2 assay (https://www.fda.gov/media/138096/download), and TaqPath COVID-19 Pooling Kit (https://www.fda.gov/media/149601/download)) or six (such as cobas SARS-CoV-2 (https://www.fda.gov/media/136049/download)) nasal swab samples could be pooled and tested together for presence of the virus, using only the resources needed for a single test. However, because the samples were diluted by their pooling, this approach increased the risk of false negative results. (See, e.g., S. A. Mahmoud et al., (2021).) Even though pool testing might prove to be a valuable way to increase surveillance for pathogens such as COVID-19, help to improve contact tracing, and reduce disruptions to society, for example by expanding testing with limited precious resources, compromised sensitivity of conventional pool testing may limit its usage due to increased false negatives. Contagious subjects may not be detected in pooled samples, which could result in failing to detect a potential outbreak or failing to accurately recognize its severity. Therefore, a method of pool testing (to expand the test volume significantly using existing precious resources) with sensitivity similar to that of individual testing (to reduce false negatives) has become an urgent need.

### SUMMARY OF THE DISCLOSURE

An objective of the present disclosure is to provide a method and a system to conduct pooling of multiple samples with high degrees of efficiency, effectiveness, and safety, and to facilitate downstream testing without the loss of sensitivity due to pooling. Another objective of this disclosure is to provide a practical solution and a commercial approach to process samples in individuals, pools, or in a combination of both pools and individual samples. A further objective of the present disclosure is to provide a solution that counteracts the disadvantages and reaps the benefits of both individual and pool testing. A further objective is to provide a method that allows for processing and testing a single starting sample of large volume. These and other objectives of the disclosure will be apparent to those skilled in the art from the description that follows.

The methods and the systems of this disclosure center around innovative work flows of sample processing, pooling, and testing.

In some embodiments, workflows include combining the processed samples, in which target is isolated or released, to make a pooled sample instead of combining the samples prior to their processing. Certain embodiments of this disclosed include, but are not limited to, systems and methods related to nucleic acid testing, i.e., identifying particular nucleic acid sequences in samples. The samples in such cases may be processed individually and then the nucleic acid (or other corresponding target of interest) is isolated. The pooled sample is created by pooling of an eluate comprising the purified nucleic acid from the individual samples. A negative pool result indicates that all samples in the pool are negative. Pools with a positive result can, in some embodiments, be de-convoluted by testing the purified nucleic acid from individual samples. The disclosed novel pooling approach allows analysis of, for instance, pools of different sample types from the same individual or, in other cases, pools of samples from different individuals.

In some embodiments, methods herein include processing multiple samples sequentially to create pooled processed samples. Certain embodiments of this disclosed include, but are not limited to, methods and systems related to nucleic acid purification, for instance using bead based technology (e.g., magnetic beads) and tests. In some cases, a first individual sample aliquot is processed to detect a particular nucleic acid sequence through hybridization to a corresponding sequence placed onto a solid surface such as beads. The beads carrying the nucleic acid of interest from the first sample may be isolated and then further added to a second sample aliquot to isolate the target nucleic acid from the second sample continuously. This process can be repeated one or more times to create pool of the bound, isolated target nucleic acid from all of the samples. A similar approach can be used to isolate other target molecules using beads, such as various small molecules, peptides, or proteins, so long as appropriate affinity beads that recognize them can be used for the assays. In this type of method, pool size does not impact target detection sensitivity as the samples are not diluted when combined into the pool.

In some embodiments, multiple samples can be processed sequentially to create a pooled, processed sample. For example, samples may be processed individually to immobilize the target molecule on beads. The target may then be eluted from the beads, and an elution buffer comprising the target from a first sample aliquot may be used to elute target from a second sample aliquot. This process can then be repeated one or more times to create a pool of eluted target from multiple samples. As in other methods herein, pool size does not impact sensitivity as the samples in the pool are not diluted.

Also disclosed is a system to combine several or all sample processing steps inside one automated system. These steps include, but are not limited to, sample storage, sample sorting, sample aliquoting, sample pooling, sample lysis, target purification, target testing, and pool de-convolution.

The following embodiments are also included herein:

Embodiment 1: A method of pooling samples for detecting the presence of a target, comprising steps of:
a) Providing a sample aliquot from each of a plurality of samples, thereby providing a plurality of sample aliquots;
b) Isolating target from each individual sample aliquot of the plurality of sample aliquots;
c) Pooling the sample aliquots after the target isolation of (b) to form a pool of sample aliquots; and
d) Testing the pool for presence of the target, whereby identification of the presence of the target in the pool indicates presence of the target in at least one aliquot of the plurality of sample aliquots.

Embodiment 2: A method of pooling samples for detecting the presence of a target, comprising steps of:
a) Providing a sample aliquot from each of a plurality of n samples, wherein n is at least two;
b) Isolating target from a first sample aliquot of the plurality;
c) Combining the first sample aliquot after the target isolation of (b) with a second sample aliquot and isolating target from the combined sample aliquots;
d) Repeating step (c) n−2 times to create a pool from the plurality of sample aliquots; and
e) Testing the pool for presence of the target, whereby identification of the presence of the target in the pool indicates presence of the target in at least one aliquot of the plurality of sample aliquots.

The method of embodiment 1 or 2, further comprising a step of determining which of the plurality of samples contains the target, comprising testing individual sample aliquots from the plurality of samples for presence of the target.

The method of any one of the preceding embodiments, wherein pooling or combining the sample aliquots after the target isolation does not result in a significant loss of sensitivity for detection of the target. In other words, in any one of the preceding embodiments, the sample aliquot may not be diluted during the pooling, combining, and isolating steps.

The method of any one of the preceding embodiments, wherein the plurality of n samples comprises at least 4 samples.

The method of the preceding embodiment, wherein the plurality of samples comprises at least 5, at least 6, at least 8, at least 10, at least 12, at least 24, at least 48, at least 96, at least 256, 4-12, 4-10, 5-10, 4-6, 6-8, 6-12, 8-12, 12-256, 24-256, 48-256, 12-96, 24-96, or 48-96 samples.

The method of any one of embodiments 1-7, wherein the target is isolated by binding to a solid surface, such as beads, resin, or membrane.

The method of the preceding embodiment, wherein pooling is performed on the solid surface, for example, by contacting target bound to the solid surface or by eluting bound target from the solid surface.

The method of the preceding embodiment, wherein the method comprises steps of:
a) Providing a sample aliquot from each of a plurality of n samples;
b) Isolating target from a first sample aliquot of the plurality by contacting the first sample aliquot with a solid surface and isolating target bound to the solid surface;
c) Contacting a second sample aliquot with the bound solid surface of (b) and isolating target bound to the solid surface;
d) Repeating step (c) n−2 times; and
e) Testing the solid surface for presence of the target, whereby identification of the presence of the target on the solid surface indicates presence of the target in at least one aliquot of the plurality of sample aliquots.

The method of any one of the preceding embodiments, wherein the method comprises steps of:
a) Providing a sample aliquot from each of a plurality of n samples;
b) Isolating target from a first sample aliquot of the plurality by contacting the first sample aliquot with a solid surface, isolating target bound to the solid surface, and eluting bound target from the solid surface to form a first eluate of the first sample aliquot;
c) Contacting a second sample aliquot with the first eluate of (b), contacting the second sample aliquot with the solid surface, isolating target bound to the solid surface, and eluting bound target from the solid surface to form a pooled eluate;
d) Repeating step (c) n−2 times; and
e) Testing the pooled eluate for presence of the target, whereby identification of the presence of the target in the pooled eluate indicates presence of the target in at least one aliquot of the plurality of sample aliquots.

The method of any one of the preceding embodiments, wherein target is isolated by enzymatic, chemical, or mechanical means.

The method of any one of the preceding embodiments, wherein at least one step of the method is automated.

The method of any one of the preceding embodiments, wherein at least two steps of the method are automated.

The method of any one of the preceding embodiments, wherein all steps of the method are automated.

The method of any one of the preceding embodiments, wherein the plurality of samples comprises samples from one individual or from one source.

The method of the preceding embodiment, wherein the plurality of samples comprises samples obtained over a range of time.

The method of any one of the preceding embodiments, wherein the plurality of samples comprises samples from more than one individual or source.

The method of any one of the preceding embodiments, wherein the samples are biologic samples, such as bodily fluid samples (e.g., blood, plasma, urine, cerebral spinal fluid, mucosa, lymph, sweat, sap, and the like), tissue samples, cell culture samples, DNA samples, RNA samples, or protein samples.

The method of any one of the preceding embodiments, wherein the samples are non-biologic samples, such as soil samples or water samples.

The method of any one of the preceding embodiments, wherein the target is a biological molecule, such as a DNA, RNA, protein, lipid, or a small molecule produced by a cell (e.g. a biological cofactor or toxin).

A system for pooling samples for detecting the presence of a target, wherein the system is capable of performing the method of any one of the preceding embodiments, wherein at least one step of the method is conducted automatically in the system.

The preceding system, which is further capable of determining which of the plurality of samples contains the target by testing individual sample aliquots from the plurality of samples for presence of the target.

The preceding system, wherein pooling of sample aliquots is performed in the system.

The system of any one of the preceding embodiments, wherein pooling the sample aliquots after the target isolation in the system does not result in a significant loss of sensitivity for detection of the target.

The system of any one of the preceding embodiments, which allows for pooling of at least 4, at least 5, at least 6, at least 8, at least 10, at least 12, at least 24, at least 48, at least 96, at least 256, 4-12, 4-10, 5-10, 4-6, 6-8, 6-12, 8-12, 12-256, 24-256, 48-256, 12-96, 24-96, or 48-96 samples.

The system of any one of the preceding embodiments, wherein the system is capable of isolating the target by binding of target to a solid surface, such as beads, resin, or membrane.

The system of the preceding embodiment, wherein the system is capable of performing sample pooling on a solid surface, for example, by contacting target bound to the solid surface or by eluting bound target from the solid surface.

The system of any one of the preceding embodiments, wherein the system processes sample aliquots in tubes or plates.

The system of any one of the preceding embodiments, wherein target is isolated within the system by enzymatic, chemical, or mechanical means.

The system of any one of the preceding embodiments, wherein at least two steps of the method are automated.

The system of the preceding embodiment, wherein all steps are automated.

The system of any one of the preceding embodiments, wherein the plurality of samples comprises samples from one individual or from one source.

The system of the preceding embodiment, wherein the plurality of samples comprises samples obtained over a range of time.

The system of any one of the preceding embodiments, wherein the plurality of samples comprises samples from more than one individual or source.

The system of any one of the preceding embodiments, wherein the samples are biologic samples, such as bodily fluid samples (e.g., blood, plasma, urine, cerebral spinal fluid, mucosa, lymph, sweat, sap, and the like), tissue samples, cell culture samples, DNA samples, RNA samples, or protein samples.

The system of any one of the preceding embodiments, wherein the samples are non-biologic samples, such as soil samples or water samples.

The system of any one of the preceding embodiments, wherein the target is a biological molecule, such as a DNA, RNA, protein, lipid, or a small molecule produced by a cell (e.g. a biological cofactor or toxin).

### BRIEF DESCRIPTION OF THE DRAWINGS

Some embodiments of the present disclosure are illustrated as an example and are not limited by the figures of the accompanying drawings, in which like references may indicate similar elements and in which:

FIG. 1—FIG. 1 illustrates the conventional way to test multiple samples individually.

FIG. 2—FIG. 2 illustrates the conventional pooling approach to combine multiple samples in pool and test pooled sample.

FIG. 3—FIG. 3 depicts a novel approach of the present disclosure to combine multiple samples without a dilution effect due to pooling multiple samples together.

FIG. 4—FIG. 4 depicts an alternative pooling approach of the present disclosure to combine multiple samples after samples are processed.

FIG. 5—FIG. 5 depicts a novel approach of the present disclosure to combine multiple samples sequentially basing on a solid based affinity mechanism.

FIG. 6—FIG. 6 depicts a novel approach of the present disclosure to pool multiple samples together. A small volume of elution buffer is used to elute target from multiple samples sequentially to create pooled sample with enriched target from multiple samples.

FIG. 7—FIG. 7 depicts a workflow to handle samples from sample loading to result in a final report.

FIG. 8—FIG. 8 depicts an automated system including modules with the function from sample loading to result in a final report.

FIG. 9—FIG. 9 depicts an exemplary schematic of a system for performing methods described herein.

FIG. 10—FIG. 10 depicts a further exemplary schematic of a system for performing methods described herein.

FIG. 11—FIG. 11 depicts a schematic of a system for performing methods described herein based on isolating a target from plates using magnetic beads.

FIG. 12—FIG. 12 depicts a further schematic of a system for performing methods described herein based on isolating a target using magnetic beads.

### DETAILED DESCRIPTION

The terminology used herein is for the purpose of describing particular embodiments only and is not intended to be limiting of the disclosure.

### Definitions

As used here, the term “and/or” includes any and all combination of one or more of the associated listed items. As used herein, the singular forms “a”, “an”, and “the” are intended to include the plural forms as well as the singular forms, unless the context clearly indicates otherwise. It will be further understood that the terms “comprises” and/or “comprising”, when used in the specification, specify the presence of stated features, steps, operations, elements, and/or components, but do not preclude the presence or addition of one or more features, steps, operations, elements, components, and/or groups thereof.

Unless otherwise defined, all terms (including technical and scientific terms) used herein have the same meaning as commonly understood by one having ordinary skill in the art to which this disclosure belongs. It will be further understood that terms, such as those defined in commonly used dictionaries, should be interpreted as have a meaning that is consistent with their meaning in the context of the relevant art and the present disclosure and will not be interpreted in an idealized or overly formal sense unless expressly so defined herein.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As utilized in accordance with the present disclosure, the following terms, unless otherwise indicated, shall be understood to have the following meanings:

A “sample” as used herein refers to any specimen that may contain a target needing detection. In some embodiments, the sample is a “biological sample,” which is a sample taken from a biological organism or source. Examples of “biological samples” include, but are not limited to, biological fluid samples, tissue samples, cell culture samples, DNA samples, RNA samples, soil samples, or water samples. A “biological fluid sample” as used herein refers to any biological fluid from an organism or subject in which may contain a target for detection. Examples include blood, plasma, urine, cerebral spinal fluid, mucosa, lymph, sweat, tears, saliva, pleural effusion, ascites, and sap. A tissue sample may contain tissue or a homogenate of tissue from any organism, such as an animal, plant, or fungus. A cell culture sample may be derived from a bacterial or eukaryotic cell culture, for example. An RNA or DNA sample may contain an RNA or DNA of interest for detection, and may, for instance, comprise an extract from another sample in which RNA or DNA is at least partially extracted or isolated. A sample may be taken directly from its source or may be pre-treated in some fashion, for example, to remove large debris or to lyse cells or to extract material of interest such as DNA or RNA.

As used herein, an “aliquot” or a “sample aliquot” means at least a portion of a sample. An aliquot may be any portion of a sample from 1% to 100% of the sample. In some embodiments, an aliquot is less than the entirety of the sample, for example, so that some of the sample remains for further individual testing. In some cases, an “aliquot” is less than 90%, less than 80%, less than 70%, less than 60%, or less than 50% of the sample.

As used herein, a “plurality” of an item, such as samples or aliquots, means more than one such item.

A “target” refers to a substance to be detected in a sample in the systems and methods herein. In some cases, a target is a nucleic acid molecule. In other cases, it is a protein or peptide. In some cases, it may be a virus. In other cases, it is a small organic molecule (i.e., a “small molecule”), such as a bacterial toxin, lipid, or a drug molecule or the like. In yet other cases, it is an inorganic molecule, e.g., a heavy metal or ion.

The term “combining” or “pooling” of two or more sample aliquots or the like simply means adding an aliquot from one sample to an aliquot from a different sample. A “pool” refers to the combined aliquots from at least two samples. In some cases, a pool of sample aliquots comprises aliquots from a much larger number of samples, such as 4, 10, 48, 96, etc.

As used herein, “isolating” a target is used in the broadest sense merely to convey at least the extraction of a target or the removal of a target from at least some contaminants. Isolating does not require additional washing, eluting, or purifying steps beyond the initial extraction of target, although, in some embodiments, such further steps may be performed. In some cases, a target may be isolated by chemical or enzymatic means, such as by enzymatic labeling. In some cases, a target may be identified by mechanical means, for example, via cell lysis, extraction of nucleic acids, or chromatography separations.

In methods herein, “testing the pool for presence of the target” is used in the broadest sense to convey any appropriate means of target detection, which may depend on the isolation method and the nature of the target (e.g., whether the target is a nucleic acid, protein, small organic molecule, inorganic molecule, etc.).

A “solid surface” is used in the broadest sense herein, and refers to a solid material such as beads (e.g., magnetic beads), resin, slurry, chips, plates, or a lipid bilayer or other membrane that may be used, for example, to bind a target or to separate a target from other molecules as part of a target isolation process.

As used herein, an “automated” or “automatically controlled” process is one that is capable of being run, for example, by a computerized control system with appropriate software, as opposed to a system that requires an active, manual intervention during or between at least one step, such as to move a sample or element from one part of the system to another. In some embodiments, the process is automated by software that controls the movements or positions of one or more components of the system, such as sample aliquots, reagents for target isolation (e.g. solid surface materials), and elements comprising isolated target. A process that is “semi-automated,” in contrast, means that at least one step but fewer than all steps are automated.

### Methods and Systems

In describing the disclosure, it will be understood that a number of techniques and steps will be disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the disclosure and the claims.

The present disclosure is to be considered as an exemplification of the methods and systems herein, and is not intended to limit the disclosure to the specific embodiments depicted by the figures or description below.

The present disclosure will now be described by referencing the appended figures representing preferred embodiments.

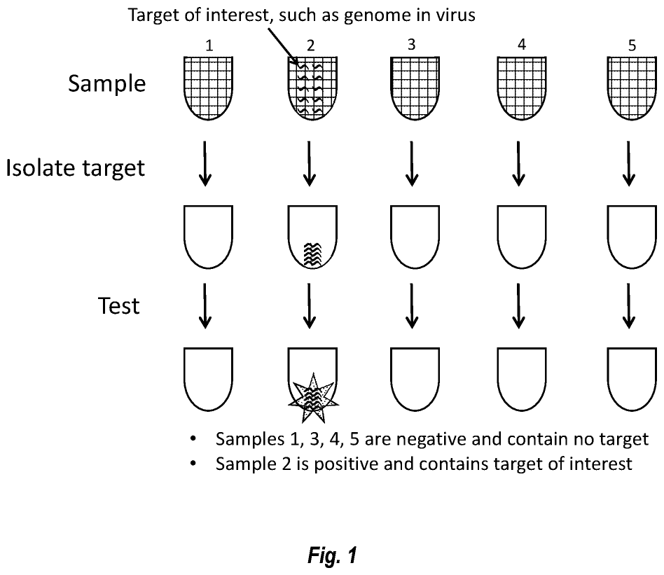

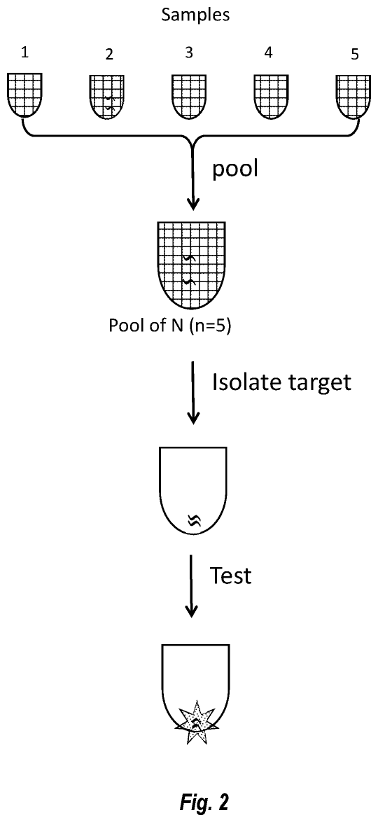

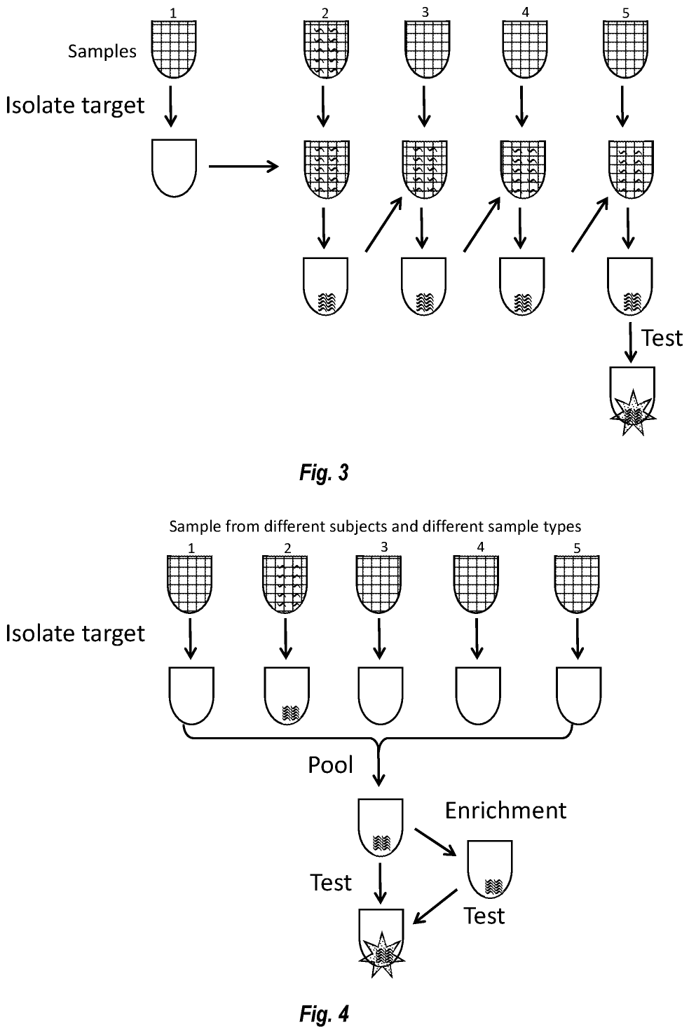

In some embodiments, the methods herein comprise a novel pooling approach as illustrated in FIG. 3. Normally, multiple samples are sampled and tested individually (FIG. 1). When large amounts of samples are required to be tested with limited precious testing kits and resources, testing pooled samples can be performed as in FIG. 2, but with the cost of lower sensitivity. For example, a positive sample containing 10 units of target per milliliter can be detected with 95% confidence by a testing method with sensitivity (95% limit of detection) of 10 units of target per milliliter. When the sample containing 10 units of target per milliliter is pooled with 4 samples containing no target, the target in the pooled sample is diluted by 5-fold to a concentration of 2 units per milliliter, which can't be detected by the testing method easily. (See FIG. 2.) In another words, for a test using 0.5 milliliter sample, only 0.1 milliliter of the individual samples in the 0.5 milliliter pool of 5 samples can be tested. Due to the associated loss of sensitivity, false negative results may occur. For a pool of 5 samples to be detected by the testing method of FIG. 2, for example, the individual samples have to contain at least 50 units per milliliter to ensure that the pooled sample contains 10 units per milliliter or more after pooling.

FIG. 3 illustrates one exemplary method herein for eliminating the loss of sensitivity that results from sample pooling. In this approach, sample aliquots are tested for presence of target prior to pooling, and tested after being pooled. For example, a sample containing 10 units of target per milliliter is processed, all target in the sample is isolated but not tested yet. The component containing the isolated target is then applied into the sample processing step of second sample. Then all targets are isolated but not tested again. The component containing the isolated target is applied into the sample processing step of next sample and the process is then repeated with all samples expected in the pool. For a pool of 5 samples, the 5^{th} sample is processed for a pool of 5 samples and tested afterwards. No dilution effect occurs in this approach (FIG. 3). In other words, when the sample containing 10 units of target per milliliter is pooled with 4 samples containing no target in this way, the target in the final pooled sample still contains 10 units of target per milliliter correspondingly, which can be detected by the testing method. Therefore, this approach avoids this sensitivity reduction and can detect target in a pooled sample with comparable sensitivity to testing the sample individually. At the same time, the approach avoids running multiple tests upon multiple samples individually which can reduce the usage of testing kits and resources by testing the pool instead. The figure illustrates a pool of five samples, but this is not intended to be limiting, and as described elsewhere herein, a pool may be derived from two samples, up to a much larger number of samples.

For example, in some embodiments, the approach described in FIG. 3 includes the following steps:
a) Providing a sample aliquot from each of a plurality of samples;
b) Isolating target from a first sample aliquot of the plurality;
c) Combining the first sample aliquot after the target isolation of (b) with a second sample aliquot and isolating target from the combined sample aliquots;
d) Optionally repeating step (c) at least once, or at least twice, depending upon the number of samples in the plurality, to create a pool from the plurality of sample aliquots; and
e) Testing the pool for presence of the target, whereby identification of the presence of the target in the pool indicates presence of the target in at least one aliquot of the plurality of sample aliquots.

Optional step (d), for example, depends upon the number of samples in the plurality. If the plurality comprises two samples, then step (d) is not necessary. If the plurality comprises three samples, step (c) is repeated, for example, in that the target isolated from the combined first and second sample aliquots is combined with a third sample aliquot and target from the further combined three sample aliquots is isolated. If the plurality comprises four samples, step (c) is repeated twice, so that the target isolated from the first and second sample aliquots is combined with that of the third sample aliquot, and target is isolated again, then the target isolated from the first, second, and third sample aliquots is combined with the fourth sample aliquot and the target is isolated once again, followed by testing the pool for presence of the target. Thus, for a plurality comprising a number of samples, n, step (d) is repeated n−2 times in order that target from all of the samples is isolated; i.e., if the number of samples is 2 then step (d) is not repeated, and if the number of samples is >2 then it is repeated. Such a method is as follows:
a) Providing a sample aliquot from each of a plurality of n samples, where n is the number of samples of the plurality;
b) Isolating target from a first sample aliquot of the plurality;
c) Combining the first sample aliquot after the target isolation of (b) with a second sample aliquot and isolating target from the combined sample aliquots;
d) Repeating step (c) n−2 times to create a pool from the plurality of sample aliquots; and
e) Testing the pool for presence of the target, whereby identification of the presence of the target in the pool indicates presence of the target in at least one aliquot of the plurality of sample aliquots.
   If the plurality of samples consists of 2 samples, then step (d) is not repeated. For any larger plurality of samples, step (d) is repeated at least once.

FIG. 4 illustrates an alternative pooling approach in some embodiments where all individual samples are processed, and the target is isolated but not tested. The target isolated from each sample is combined to create a pool, and the pooled, processed sample is tested. In some embodiments, the pooled, processed sample is processed again before being tested in order to concentrate the target, for example, by resuspending or eluting the target in a small volume of solution, to further improve the method sensitivity. This approach avoids the sensitivity reduction of traditional pooling (e.g. FIG. 2) and can further improve sensitivity over testing the samples individually. Meanwhile, this approach avoids running multiple tests upon multiple samples individually and reduces the usage of testing kits and precious resources by testing a sample pool instead.

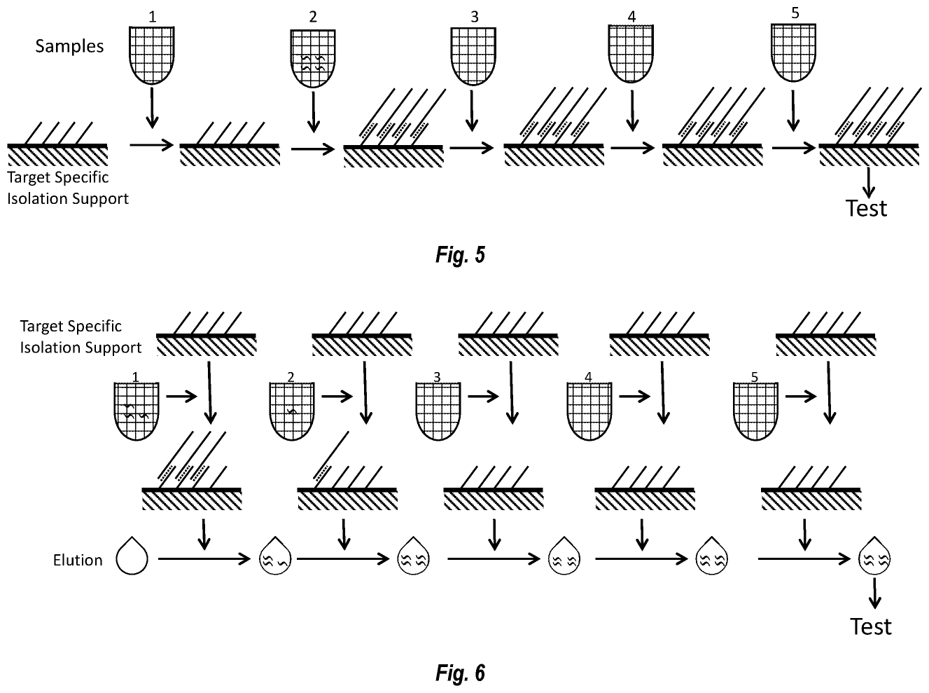

FIG. 5 illustrates an exemplary method to fulfill the novel pooling approach depicted in FIG. 3, utilizing a solid surface as a means to isolate the target. A solid surface with affinity to target (such as magnetic beads, resin, lipid membrane, etc., that comprise or are coated with a molecule that specifically recognizes the target), is applied to isolate the target from samples. If needed, the target may first be released or extracted using chemical, enzymatic, mechanical, or other methodologies, then is isolated by exposure to the solid surface. The solid surface is then used to bind the target that was released from the sample. In this approach, the first sample is processed to the solid surface to allow the target to bind to the solid surface. The solid surface then is isolated from the first sample. For example, beads or chips may be isolated by centrifugation or precipitation from a liquid sample. Molecules that do not bind specifically to the solid surface may be removed, for example, as a supernatant, or after one or more wash steps. And then, the next (second) sample is exposed to the target-bound solid surface, and any target from the second sample is then allowed to bind to the solid surface, and the bound solid surface is collected for a second time. This process can then be repeated with all samples in the pool. After the last sample has been processed in this way, the resulting solid surface is tested for presence of target. If no target is present, then it can be concluded that none of the individual samples contains target sufficient to be detectable in the assay. If target is detected, then it can be concluded that at least one sample from the pool contains target. No dilution effect occurs in this approach (FIG. 5). Therefore, this approach avoids the sensitivity reduction and can detect samples being comprised in pool with the comparable sensitivity to testing the same samples individually. At the same time, the approach avoids running multiple tests upon multiple samples individually and reduces the usage of testing kits and resources by testing pool instead, and allows large pool size without significant sensitivity loss.

For example, in some embodiments, the approach of FIG. 5 includes the following steps:
a. Providing a sample aliquot from each of a plurality of samples;
b. Isolating target from a first sample aliquot of the plurality by contacting the first sample aliquot with a solid surface and isolating target bound to the solid surface;
c. Contacting a second sample aliquot with the bound solid surface of (b) and isolating target bound to the solid surface;
d. Optionally, repeating step (c) at least once or at least twice, or n−2 times where n=the number of samples in the plurality; and
e. Testing the solid surface for presence of the target, whereby identification of the presence of the target on the solid surface indicates presence of the target in at least one aliquot of the plurality of sample aliquots.
   In some embodiments herein, the pooled, solid surface of (e) is resuspended in a buffer of lower volume than that of each sample aliquot, thus further concentrating the target to be detected.

FIG. 6 illustrates another method to fulfill the novel pooling approach. Multiple samples are processed to release target and bind target to a solid surface. Optionally at least one wash step is performed to remove non-specifically bound molecules from the surface. A specific volume of elution buffer is applied to the solid surface to elute the target from the first sample. Further solid surface is then put into contact with the next (e.g., second) sample aliquot, and the process is repeated with that sample aliquot, except that the elution buffer containing target from the first sample aliquot is used as the elution buffer for the next (second) sample aliquot, and so on, so that once all samples are processed, the final elution buffer contains all isolated target from the various samples and is, in effect, a pooled elution of target from all of the samples. This pooled elution is then tested for presence of the target. No dilution effect occurs in this approach (FIG. 6). Therefore, this approach, as with those described in FIGS. 3-5 above, avoids the sensitivity reduction of traditional pooling and can be used to detect target in samples with comparable sensitivity to testing the samples individually. At the same time, the approach avoids running multiple tests upon multiple samples individually and reduces the usage of testing kits and resources by testing pool instead, and allows large pool size without significant sensitivity loss. For example, in some cases, after washing and elution, a solid surface may be used again for the next sample in the pool and, in some cases, may be re-used multiple times, thus reducing the amount of the solid surface. This may be important, for example, if the solid surface containing affinity reagent for the target is difficult or expensive to produce, as can often be the case for unique target molecules.

In embodiments as illustrated in FIG. 6, multiple samples can also be processed using different methodologies. The isolated target from multiple samples is combined to create the pool. The pooled target from multiple samples can then be tested. This approach allows different sample types, which requires different sample processing technologies, to be pooled. In some cases, the pooled target from multiple samples can be processed again to concentrate target into a smaller volume than that of the original sample aliquots, which allows detection of very low concentration of target from individual samples or large pools, and may further increase the sensitivity of the methods.

In some embodiments, a method as shown in FIG. 6 may be conducted as follows, with steps comprising:
a) Providing a sample aliquot from each of a plurality of samples;
b) Isolating target from a first sample aliquot of the plurality by contacting the first sample aliquot with a solid surface, isolating target bound to the solid surface, and eluting bound target from the solid surface to form a first eluate of the first sample aliquot;
c) Contacting a second sample aliquot with the first eluate of (b), contacting the second sample aliquot with the solid surface, isolating target bound to the solid surface, and eluting bound target from the solid surface to form a pooled eluate;
d) Optionally, repeating step (c) at least once, at least twice, or n−2 times where n=the number of samples in the plurality; and
e) Testing the pooled eluate for presence of the target, whereby identification of the presence of the target in the pooled eluate indicates presence of the target in at least one aliquot of the plurality of sample aliquots.

In some embodiments herein, the eluate from each sample has a lower volume than the original sample aliquot used in steps (b) and (c). In some embodiments, herein, prior to testing the pooled eluate for presence of the target, the pooled eluate is processed to reduce its volume and increase its concentration. For example, it may be passed through a filter that retains target and re-suspended in a smaller volume, or it may be treated to remove solvent, etc.

In some embodiments herein, the plurality of samples to be tested in series according to the methods is at least 2. In some embodiments herein, the plurality of samples to be tested in series according to the methods is at least 3. In some embodiments herein, the plurality of samples to be tested in series according to the methods is at least 4. In some cases, it is at least 5 at least 6, at least 8, or at least 10, at least 12, at least 24, at least 48, at least 96, or at least 256. In some cases, the plurality comprises 2-12, 4-12, 4-10, 5-10, 4-6, 6-8, 6-12, 8-12 samples. In other cases, the plurality comprises 12-256, 24-256, 48-256, 12-96, 24-96, 48-96 samples. In some cases, the plurality of samples may be provided on a microtiter plate for example, comprising 12, 24, 48, 96, or 256 wells or holders for individual sample cartridges, and some or all of the wells or positions on the plate may be used to hold the plurality of samples for pooling and testing.

In some embodiments, creation of the pool of sample aliquots does not effectively dilute any of the sample aliquots, in contrast to creation of a traditional sample pool. In other words, each sample aliquot can be serially processed to isolate target from its full volume. For example, if each sample aliquot is 1 mL and if this is the volume that can be practically used for testing, creation of a traditional pool of 4 sample aliquots would require taking a volume of 250 mL from each of the 4 so that the pooled sample for testing is 1 mL. In the present method, each 1 mL sample aliquot is processed serially in its full volume. Thus, in some embodiments, sample aliquots are not diluted when forming the pool.

The number of samples in the plurality may depend, in some cases, on the number of samples that are expected initially to contain target. For example, if it is estimated that about 1 in 4 samples will contain target, a small plurality of samples, such as only 2 or 3, may be sufficient for pooling since, a pool of 4 or larger would be expected to be 100% positive on average. If it is estimated that about 1 in 10 samples will contain target, then a larger plurality of samples can be pooled together. If it is estimated that about 1 in 1000 to 1 in 1,000,000 samples will contain target, then significantly larger pluralities of samples may be pooled together, such as using microtiter plates as described above for pools of 10, 24, 48, 96, or 256 samples, for instance.

In some embodiments, if the method shows presence of target, then further testing is conducted to determine which sample or samples of the plurality contain target. For example, individual sample aliquots from each member of the plurality may be re-tested for presence of the target. Alternatively, a larger pool may be deconvoluted by first testing smaller sub-pools for presence of target and then testing individual sample aliquots from any positive sub-pools.

In some embodiments, samples in a plurality comprise barcodes. In other embodiments, the samples are not barcoded.

In some embodiments herein, at least one step of the method is automated. In other cases, at least two steps are automated. In yet other cases, all steps are automated.

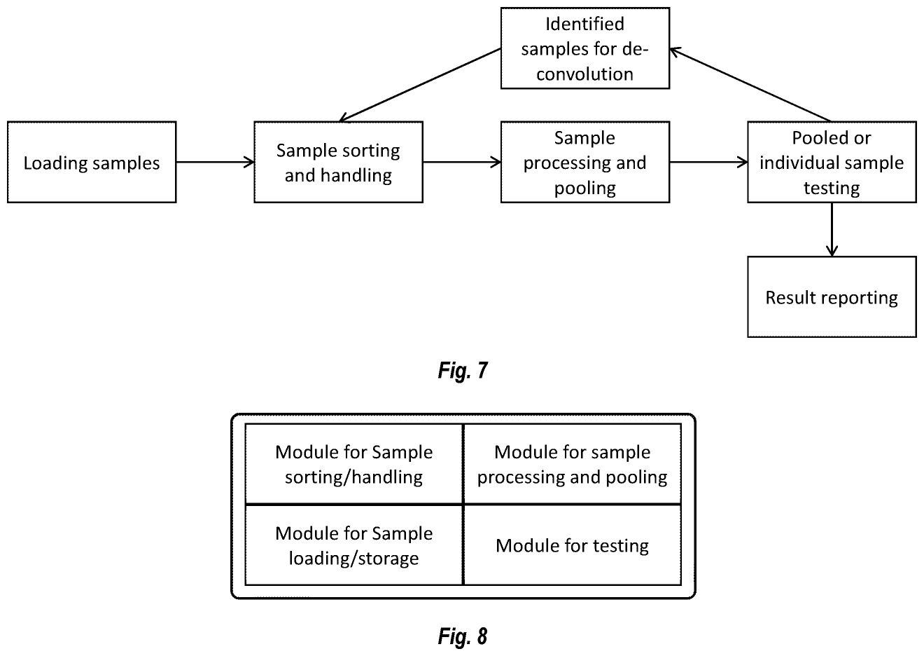

FIG. 7 illustrates an example of workflow, which allows individual or pooled samples to be prepared and tested with similar sensitivity. An automated system to fulfill the workflow is illustrated in FIG. 8. Partial workflow and system can be organized to include different function modules for needed functionalities.

Aspects of the present disclosure are also embodied in systems, apparatuses, and processes that complete multiple functionalities being required by a diagnostic system. In some exemplary embodiments, a diagnostic system can be configured to perform sample sorting/storage, de-capping/capping, aliquot preparation, sample pooling, sample processing, target isolation, sample analyzing, and de-convoluting of pooled samples. In some cases, a diagnostic system comprises at least one functional module to automate processing of one or multiple functions. The functional module can be added and/or extended to add functionalities. The functional module or modules can be assembled inside a main unit of the system or can be connected to the unit either manually or via automation.

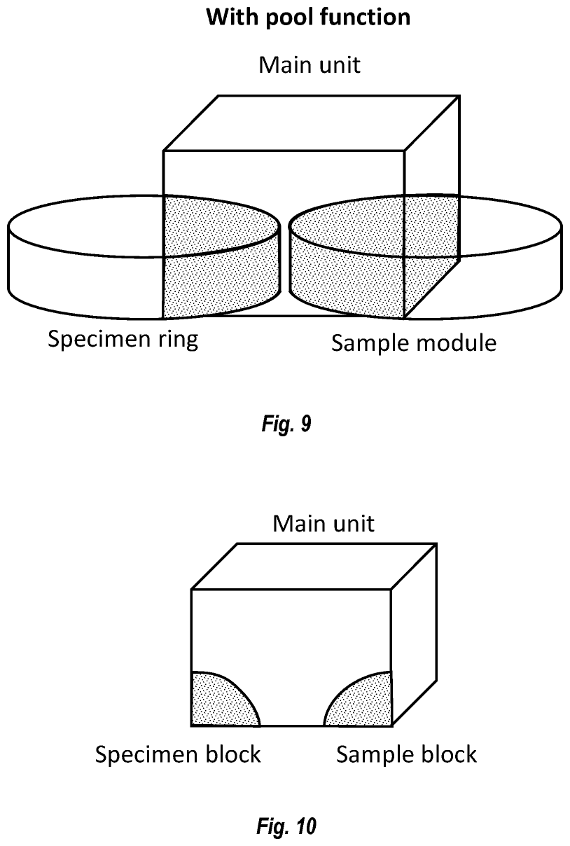

In some exemplary embodiments, a system can be configured to include, for example, a specimen module, sample module, and main unit (FIG. 9). The specimen module may be designed to support loading and storage of samples, for example, for instance in tubes or other appropriate containers. The sample module may be designed to provide tubes or other containers to hold the aliquots of the samples and support sample pooling processes. The main unit may be designed to complete functions such as de-capping/capping of tubes, moving of samples or aliquots to tubes, plates, or other containers, combining multiple sample aliquots to make pools, processing of pools, isolating and detecting a target of interest, and tracking sample and reporting results.

In other embodiments, the system performs various functions, but the main unit does not combine multiple sample aliquots to make pools. An example of such a specimen module, sample module, and main unit is provided, for example, in FIG. 10. In that case, the specimen module may be designed to support loading and storage of samples, for example, for instance in tubes or other appropriate containers. The sample module may be designed to provide tubes or other containers to hold the aliquots of the samples and support sample pooling processes. The main unit may be designed to complete functions such as de-capping/capping of tubes, moving of samples or aliquots to tubes, plates, or other containers, isolating and detecting a target of interest, and tracking sample and reporting results.

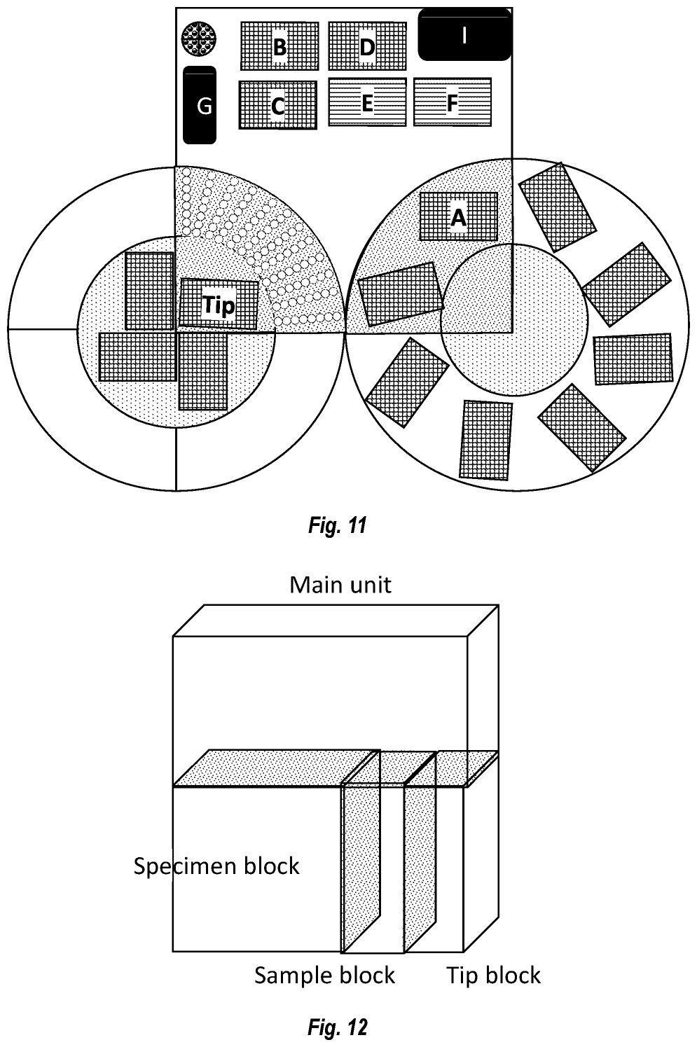

In some embodiments, a system can be designed based on the isolation of a target using magnetic beads (FIG. 11). The specimen module in such a case may be configured to include sample container loading and storage, and pipette tips to support individual sample handling, such as removing aliquots of samples to another container, adding reagents to samples, etc. The sample module may be configured in some embodiments, as shown in FIG. 11, to use plates to hold sample aliquots. A plate may contain sample processing liquid to isolate a target of interest, e.g., a cell lysis buffer to isolate a target found in cells, or other reaction buffer to isolate a target from other, contaminating molecules. Magnetic beads may be included in the plate A, for example, to absorb the target of interest. The beads carrying the target of interest may be washed and processed further in the main module, and the targets may be amplified and detected thereafter. In other exemplary embodiments, magnetic beads may be moved from plate A to the next sample plate B using a magnetic field to isolate the target of interest from combined plates A and B. The process may be repeated n times, where n is the number of samples minus 2, to create a pool of target absorbed on the magnetic beads. The beads may then be washed and processed further in the main module and any target bound to the beads detected thereafter. In another exemplary embodiment, the multiple function modules of such a system may be designed and configured to establish a stand-alone system (FIG. 12).

As noted elsewhere, methods and systems herein are also amenable to a wide variety of sample types and methods for isolating and detecting target molecules. For example, a sample may be biologic or non-biologic. A target may be an organic/biologic molecule, such as a protein, peptide, DNA, RNA, lipid, cofactor, toxin, hormone, or the like. Or it may be a drug molecule or its metabolite. Or it may be an inorganic molecule such as a heavy metal or heavy metal ion. A target may also be a virus, an inorganic particle (e.g. from soil), or the like. A sample may be, for instance, a bodily fluid sample (e.g. blood, plasma, urine, cerebral spinal fluid, mucosa, lymph, sweat, tears, ascites, pleural effusion from an individual or sap from a tree), tissue sample, or a laboratory sample such as a cell culture sample, DNA sample, RNA sample, or the like. A sample may also be a soil or water sample (e.g., to search for presence of toxic chemicals or harmful viruses, bacteria, fungi, or protozoa, and the like). In some cases, a water sample could be a sewage sample or a sample from run-off into a lake, pond, stream, river, ocean, etc. A sample may also be from a product such as a drug solution (e.g. to look for degradation product targets or contaminants) or other commercially sold product such as a food product or cosmetic product (e.g. to look for toxins or contaminants). In some cases, the plurality of samples may have different sources (e.g. biologic samples from more than one individual; soil samples from different locations). In other cases, the plurality of samples may all be from a single source, but, for example, taken at different times or under different conditions. In yet other cases, the plurality of samples may be multiple aliquots taken from one, larger sample, and the method may be used, for example, to obtain a target from the sample as a whole by splitting it into a plurality of smaller samples for pooling.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the scope of the present disclosure, are contemplated thereby, and are intended to be covered by the following claims. References cited herein are incorporated in their entirety into the disclosure.

What is claimed is:

1. A method of pooling samples for detecting the presence of a target, comprising steps of:
a. Providing a sample aliquot from each of a plurality of samples, thereby providing a plurality of sample aliquots;
b. Isolating target from each individual sample aliquot of the plurality of sample aliquots;
c. Pooling the sample aliquots after the target isolation of (b) to form a pool of sample aliquots; and
d. Testing the pool for presence of the target, whereby identification of the presence of the target in the pool indicates presence of the target in at least one aliquot of the plurality of sample aliquots.

2. A method of pooling samples for detecting the presence of a target, comprising steps of:
a. Providing a sample aliquot from each of a plurality of n samples, wherein n is at least two;
b. Isolating target from a first sample aliquot of the plurality;
c. Combining the first sample aliquot after the target isolation of (b) with a second sample aliquot and isolating target from the combined sample aliquots;
d. Repeating step (c) n−2 times to create a pool from the plurality of sample aliquots; and
e. Testing the pool for presence of the target, whereby identification of the presence of the target in the pool indicates presence of the target in at least one aliquot of the plurality of sample aliquots.

3. The method of claim 1 or 2, further comprising a step of determining which of the plurality of samples contains the target, comprising testing individual sample aliquots from the plurality of samples for presence of the target.

4. The method of any one of claims 1-3, wherein pooling or combining the sample aliquots after the target isolation does not result in a significant loss of sensitivity for detection of the target.

5. The method of any one of claims 1-5, wherein the plurality of n samples comprises at least 4 samples.

6. The method of claim 6, wherein the plurality of samples comprises at least 5, at least 6, at least 8, at least 10, at least 12, at least 24, at least 48, at least 96, at least 256, 4-12, 4-10, 4-6, 6-8, 6-12, 8-12, 12-256, 24-256, 48-256, 12-96, 24-96, or 48-96 samples.

7. The method of any one of claims 1-7, wherein the target is isolated by binding to a solid surface, such as beads, resin, or membrane.

8. The method of claim 8, wherein pooling is performed on the solid surface, for example, by contacting target bound to the solid surface or by eluting bound target from the solid surface.

9. The method of claim 9, wherein the method comprises steps of:
a. Providing a sample aliquot from each of a plurality of n samples;
b. Isolating target from a first sample aliquot of the plurality by contacting the first sample aliquot with a solid surface and isolating target bound to the solid surface;
c. Contacting a second sample aliquot with the bound solid surface of (b) and isolating target bound to the solid surface;
d. Repeating step (c) n−2 times; and
e. Testing the solid surface for presence of the target, whereby identification of the presence of the target on the solid surface indicates presence of the target in at least one aliquot of the plurality of sample aliquots.

10. The method of claim 6, wherein the method comprises steps of:
a. Providing a sample aliquot from each of a plurality of n samples;
b. Isolating target from a first sample aliquot of the plurality by contacting the first sample aliquot with a solid surface, isolating target bound to the solid surface, and eluting bound target from the solid surface to form a first eluate of the first sample aliquot;
c. Contacting a second sample aliquot with the first eluate of (b), contacting the second sample aliquot with the solid surface, isolating target bound to the solid surface, and eluting bound target from the solid surface to form a pooled eluate;
d. Repeating step (c) n−2 times; and
e. Testing the pooled eluate for presence of the target, whereby identification of the presence of the target in the pooled eluate indicates presence of the target in at least one aliquot of the plurality of sample aliquots.

11. The method of any one of claims 1-11, wherein target is isolated by enzymatic, chemical, or mechanical means.

12. The method of any one of claims 1-12, wherein at least one step of the method is automated.

13. The method of any one of claims 1-12, wherein at least two steps of the method are automated.

14. The method of any one of claims 1-12, wherein all steps of the method are automated.

15. The method of any one of claims 1-15, wherein the plurality of samples comprises samples from one individual or from one source.

16. The method of claim 16, wherein the plurality of samples comprises samples obtained over a range of time.

17. The method of any one of claims 1-15, wherein the plurality of samples comprises samples from more than one individual or source.

18. The method of any one of claims 1-18, wherein the samples are biologic samples, such as bodily fluid samples (e.g., blood, plasma, urine, cerebral spinal fluid, mucosa, lymph, sweat, sap, and the like), tissue samples, cell culture samples, DNA samples, RNA samples, or protein samples.

19. The method of any one of claims 1-18, wherein the samples are non-biologic samples, such as soil samples or water samples.

20. The method of any one of claims 1-20, wherein the target is a biological molecule, such as a DNA, RNA, protein, lipid, or a small molecule produced by a cell (e.g. a biological cofactor or toxin).

21. A system for pooling samples for detecting the presence of a target, wherein the system is capable of performing the method of any one of claims 1-21, wherein at least one step of the method is conducted automatically in the system.

22. The system of claim 22, which is further capable of determining which of the plurality of samples contains the target by testing individual sample aliquots from the plurality of samples for presence of the target.

23. The system of claim 22 or 23, wherein pooling of sample aliquots is performed in the system.

24. The system of any one of claims 22-24, wherein pooling the sample aliquots after the target isolation in the system does not result in a significant loss of sensitivity for detection of the target.

25. The system of any one of claims 22-25, which allows for pooling of at least 4, at least 5, at least 6, at least 8, at least 10, at least 12, at least 24, at least 48, at least 96, at least 256, 4-12, 4-10, 5-10, 4-6, 6-8, 6-12, 8-12, 12-256, 24-256, 48-256, 12-96, 24-96, or 48-96 samples.

26. The system of any one of claims 22-26, wherein the system is capable of isolating the target by binding of target to a solid surface, such as beads, resin, or membrane.

27. The system of claim 27, wherein the system is capable of performing sample pooling on a solid surface, for example, by contacting target bound to the solid surface or by eluting bound target from the solid surface.

28. The system of any one of claims 22-28, wherein the system processes sample aliquots in tubes or plates.

29. The system of any one of claims 22-28, wherein target is isolated within the system by enzymatic, chemical, or mechanical means.

30. The system of any one of claims 22-28, wherein at least two steps of the method are automated.

31. The system of claim 31, wherein all steps are automated.

32. The system of any one of claims 22-32, wherein the plurality of samples comprises samples from one individual or from one source.

33. The system of claim 33, wherein the plurality of samples comprises samples obtained over a range of time.

34. The system of any one of claims 22-34, wherein the plurality of samples comprises samples from more than one individual or source.

35. The system of any one of claims 22-35, wherein the samples are biologic samples, such as bodily fluid samples (e.g., blood, plasma, urine, cerebral spinal fluid, mucosa, lymph, sweat, sap, and the like), tissue samples, cell culture samples, DNA samples, RNA samples, or protein samples.

36. The system of any one of claims 22-35, wherein the samples are non-biologic samples, such as soil samples or water samples.

37. The system of any one of claims 22-37, wherein the target is a biological molecule, such as a DNA, RNA, protein, lipid, or a small molecule produced by a cell (e.g. a biological cofactor or toxin).
